(19) United States
(12) Patent Application Publication
(10) Pub. No.: US 2004/0110629 A1
(43) Pub. Date: Jun. 10, 2004

(54) Catalyst for the production of light olefins

(76) Inventors: Dennis Stamires, Newport Beach, CA (US); Paul O'Connor, Hoevelaken (NL); Arja Hakuli-Pieterse, Alkmoor (NL); Rajeev S. Rao, Webster, TX (US); Erik Jeroen Laheij, Amstelveen (NL)

Correspondence Address: LOUIS A. MORRIS, AKZO NOBEL INC., 7 LIVINGSTONE AVENUE, DOBBS FERRY, NY, 10522-3408, US

(21) Appl. No.: 10/650,313

(22) Filed: Aug. 28, 2003

Related U.S. Application Data
(60) Provisional application No. 60/407,223, filed on Aug. 29, 2002.

(51) Int. Cl. C10G 11/02; C07C 4/06; B01J 29/04

(52) U.S. Cl. 502/60; 208/113; 208/120.01; 585/653

(57) ABSTRACT

The invention comprises a catalyst composition comprising a pentasil type of zeolite, one or more solid acidic promoters and optionally a filler and/or binder, methods for making the catalyst composition and a process for using the catalyst in the manufacture of olefins.

### CROSS REFERENCE TO RELATED APPLICATION

This application claims priority from U.S. Patent Application No. 60/407,223, filed Aug. 29. 2002.

### BACKGROUND OF THE INVENTION

1. Field of the Invention

The present invention is related to the catalytic production of light olefins.

2. Prior Art

In recent years, there has been a tendency to utilize the fluid catalytic cracking process, not as a gasoline producer, but as a process to make light olefins for use as petrochemical materials or as building blocks for gasoline blending components, such as MTBE and alkylate.

The traditional method for the production of light olefins, such as ethylene, propylene, and butylene, from petroleum hydrocarbon is tubular furnace pyrolysis or pyrolysis over heat carrier or by catalytic conversion of lower aliphatic alcohol. More recently, the fluid catalytic cracking process employing small pore zeolite additives from the pentasil family is being used for the same at modern refinery. The small pore zeolite additives can be prepared as described in several patents (e.g. U.S. Pat. No. 5,472,594, or WO98/41 595).

Further descriptions of the production of light olefins by cracking processes are given in U.S. Pat. No. 3,541,179; and JP No. 60-222 428.

The small pore zeolite additives are applied at the refinery by blending with the FCC host catalyst typically at 1-5 wt-% concentration. The obtained light olefin increase depends on the effectiveness of the additive, on the base catalyst formulation, feed type, and FCC process conditions, such as residence time and temperature. However, if the refiner targets a light olefin concentration, which is higher than that obtained at 1-5 wt-% intake of the small pore zeolite additive, usually the overall performance will start to deteriorate. This is because of a dilution of the host catalyst and increase in the bottoms conversion and saturation of the light olefins yield.

### SUMMARY OF THE INVENTION

In one embodiment, the present invention is a catalyst composition comprising a pentasil type of zeolite, one or more solid acidic promoters and, optionally, a filler and/or binder.

In a second embodiment, the present invention is a method of making the above catalyst composition, wherein an aqueous slurry comprising the pentasil-type zeolite and solid acidic cracking promoter is prepared and dried.

In a third embodiment, the present invention is a process for producing olefins having up to about 6 carbon atoms per molecule, comprising contacting a petroleum feedstock at fluid catalytic cracking conditions with the above catalyst composition.

Other embodiments of the invention relate to details concerning catalyst composition, making the catalyst composition and use of the composition in making olefins.

### DETAILED DESCRIPTION OF THE INVENTION

The present invention describes FCC catalyst and catalyst/additive systems, which can be used to produce higher concentrations of olefins, particularly propylene, than obtained with the conventional additive systems as described above, and at the same time achieving high bottoms conversion. The systems are designed to function also in the processing of heavier feeds, which are especially sensitive to the dilution effects when using the conventional catalyst/additive systems at higher additive concentrations. The systems of this invention do not suffer from the dilution of the active ingredients and deterioration of the overall performance.

Particular achievements of the invention are:

Effective ex-situ stabilization and/or modification of the small pore zeolite(s) in an additive/host and in catalyst particle system, in the presence of other active catalyst ingredients.

Design of the additive/host and one particle catalyst system, which are highly active in upgrading the bottoms in gasoline and gas. The upgraded gasoline components are olefinic in nature. The active ingredients of the catalyst composition are selected in such a way that occurrence of hydrogen transfer and aromatization reactions, which are detrimental to the production of light olefins, are minimized.

The additive/host or the one particle system, as prepared according to this patent, exhibits high bottoms conversion, in particular when very high quantities of the small pore zeolite are used in the blend.

The present invention describes catalyst compositions which exhibit improved activities and selectivities, as compared to the catalysts described in the prior art, for producing higher yields of light olefins, LCO, and gasoline, with minimum activities for hydrogen transfer reactions.

This invention involves the use of certain modified forms of pentasil-type zeolites (metalloaluminosilicates) components together with one or more acidic cracking promoter components with the option of including binders, fillers, extenders, etc., incorporated in a catalytic particle.

In contrast to the prior art, this invention does not depend on the use of traditional Rare Earth exchanged zeolite Y (REY, REHY, REUSY, REMgY) used in commercial FCC products. Use of these zeolites decreases olefin yields because of the high hydrogen transfer reaction activities.

Catalyst Composition of the Invention

As stated above, the catalyst composition of the invention comprises a pentasil-type of zeolite, one or more solid acidic cracking promoters and, optionally, a filler and/or binder.

The pentasil-type of zeolite may comprise:

zeolite selected from the group consisting of ITQ-type zeolite, beta zeolite and silicalite;

ZSM-type zeolite;

pentasil zeolite doped with a compound comprising a metal ion selected from the group consisting of ions of alkaline earth metals, transition metals, rare earth metals, phosphorous, boron, aluminum, noble metals and combinations thereof; or

crystals having metals in tetrahedral coordination in the crystals selected from the group consisting of Al, As, B, Be, Co, Cr, Fe, Ga, Hf, In, Mg, Mn, Ni, P, Si, Ti, V, Zn, Zr and mixtures thereof.

The solid acidic cracking promoter in the catalyst composition of the invention may be selected from the group consisting of alumina modified by incorporation of acid centers thereon or therein, acidic silica-alumina co-gels, acidic natural or synthetic clays, acidic titania, acidic zirconia, acidic titania-alumina, acidic zeolite materials and co-gels of titania, alumina, zirconia, phosphates, borates, aluminophosphates, tungstates, molybdates and mixtures thereof. The acid centers may be selected from the group consisting of halides, sulfates, nitrates, titanates, zirconates, phosphates, borates, silicates and mixtures thereof. The solid acidic cracking promoter may comprise acidic silica-alumina, titania-alumina, titania/zirconia, alumina/zirconia or aluminum phosphate co-gels modified by the incorporation therein of metal ions or compounds selected from the group consisting of alkaline earth metals, transition metals, rare earth metals and mixtures thereof. The acidic silica-alumina co-gels may have been subjected to hydrothermal treatment.

The acidic natural or synthetic clays may have been modified by calcining, steaming, dealumination, desilification, ion exchange, pillaring exfoliation or combinations thereof.

The acidic titania, acidic zirconia, or both may be doped with sulfates, vanadates, phosphates, tungstates, borates, iron, rare earth metals or mixtures thereof.

The acidic zeolite materials may be selected from the group consisting of mordenite, zeolite Beta, NaY zeolite and USY zeolite that is dealuminated or ion exchanged with transition metals or both. The preferred transition metal is vanadium.

In the catalyst composition of the invention, the solid acidic cracking promoter may comprise a co-gel of alumina-aluminum-phosphate or aluminum phosphate that has been doped with an acidic compound.

The catalyst composition of the invention may comprise one or more additional materials selected from the group consisting of particle binders, diluents, fillers and extenders. The pentasil-type zeolite is a pentasil type of zeolite may comprise from about 5.0 wt % to about 80 wt % of the composition. The composition may comprise particles having average lengths along their major axis of from about 30 microns to about 150 microns.

The weight ratio of said pentasil-type zeolite to solid acidic cracking promoter in the catalyst composition of of the invention may be from about 0.03 to about 9.0.

The solid acidic cracking promoter in the composition may comprise from about 5.0 wt % to about 80 wt % of the composition.

The catalyst composition of the invention may comprise particles having average lengths along their major axis of from about 20 microns to about 200 microns.

The Modified Forms of Pentasil-Type Zeolite

For clarity and simplicity, and to distinguish from the ZSMs known in the art, the modified pentasil zeolites, prepared according to this invention are identified as MPZ-(ZSMs). For example, some of the types of pentasil zeolites used in the invention involve, but are not limited to, ZSM-5, ZSM-11, ZSM-12, ZSM-22, ZSM-23, ZSM-35, zeolite beta, zeolite boron beta, which are described in U.S. Pat. Nos. 3,308,069; 3,702,886; 3,709,979; 3,832,449; 4,016,245; 4,788,169; 3,941,871; 5,013,537; 4,851,602; 4,564,511; 5,137,706; 4,962,266; 4,329,328; 5,354,719; 5,365,002; 5,064,793; 5,409,685; 5,466,432; 4,968,650; 5,158,757; 5,273,737; 4,935,561; 4,299,808; 4,405,502; 4,363,718; 4,732,747; 4,828,812; 5,466,835; 5,374,747; 5,354,875; incorporated herein by reference. Metals in tetrahedra coordination in the zeolite crystals include: AL, AS, B, Be, Co, Cr, Fe, Ga, Hf, In, Mg, Mn, Ni, P, Si, Ti, V, Zn, Zr.

Modified forms of pentasil-type zeolites (here and after referred to as MPZs) such as ZSMs, Beta and so on, briefly involve doping said zeolites with metal ions such as, but not limited to alkaline earth, transition metals, rare earth metals, phosphorous, boron, aluminum. The MPZ zeolites can be mixed with regular pentasil zeolites (i.e., ZSM, Beta, etc.) or with ion exchanged forms of pentasil zeolites, known to present state of the art such as pentasil zeolites exchanged with transition metals.

The pentasil zeolite may be doped with a compound comprising a metal ion selected from the group consisting of ions of alkaline earth metals, transition metals, rare earth metals, phosphorous, boron, aluminum, noble metals and combinations thereof. The pentasil-type zeolite may be doped by any of the following methods:

ion exchange with the metal ion;

use of doped seeds;

use of doped reactants;

use of seeds comprising X- or Y-type zeolites that have never been ion exchanged with the metal ion;

incorporating salts comprising the metal ion in a reaction mixture comprising the precursor of the pentasil-type zeolite.

Making the Catalyst of the Invention

In making the catalyst composition of the invention an aqueous slurry comprising a pentasil-type zeolite and solid acidic cracking promoter is prepared and dried. Separate aqueous slurries of the pentasil-type zeolite and solid acidic cracking promoter may be prepared, mixed together and dried. The aqueous slurry may be spray dried to obtain catalyst particles having average lengths along their major axis of from about 40 microns to about 100 microns.

The catalyst composition of the invention may comprise one or more additional materials selected from the group consisting of particle binders, diluents, fillers and extenders. This may be made by modifying pentasil-type zeolite by ion exchange with ions selected from the group consisting of ions of alkaline earth metals, transition metals, rare earth metals, phosphorous, boron, aluminum, noble metals and combinations thereof, preparing an aqueous slurry of acidic cracking promoter and other catalyst ingredients other than the modified pentasil-type zeolite, adding the modified pentasil-type zeolite to the slurry and shaping the slurry, the addition of the modified pentasil-type zeolite being carried out as a final step immediately prior to shaping. The addition of the modified pentasil-type zeolite may be carried out by mixing with thed aqueous slurry until the slurry is substantially homogeneous. Shaping may be carried out by spray drying.

NH₄OH may be added to the slurry prior to the addition of the modified pentasil-type zeolite to raise the pH of the slurry. A pH buffer may be added to the slurry prior to the addition of the modified pentasil-type zeolite. The buffer may be selected from the group consisting of aluminum chlorohydrol, phosphate sol or gel, anionic clay, smectite and thermally or chemically modified clay. The thermally or chemically modified clay imay be kaolin clay.

An aqueous slurry may be prepared comprising solid acidic cracking promoter and precursors of the pentasil-type zeolite comprising silica, alumina and seeds containing one or more metals from the group consisting of rare earth metals, alkaline earth metals and transition group metals, forming the aqueous slurry into shaped bodies and crystallizing the pentasil-type zeolite in situ in the shaped body.

The Acidic Cracking Promotor Components

Referred to hereinafter as ACPs, these are solid acidic materials which provide an additional higher acidic function to the catalytic cracking particle which supplements the function of the pentasil zeolite component and synergistically through the cracking process produce higher yields of light olefins (i.e., ethylene, propylene, butylene, and pentenes).

There is a large number of solid acids known in the state of the art, of which a few are described below to illustrate the scope of this invention; however, this invention is not thereby limited.

Some of the ACPs involve solid acids, solid super acids, acidic zeolites such as hydrogen modernite, dealuminated Y zeolites such as DAYs, high SAR USY dealuminated zeolites used in hydrocracking, aluminum exchanged zeolites, LZ-210, USY aluminum exchanged, transition metal ion exchanged Y, USY, DAY zeolites, alumina containing acidic ions, silica-alumina exchanged with acidic ions, titania-alumina containing acidic ions, titania-zirconia containing acidic ions, alumina-zirconia containing acidic ions, alumina-aluminum phosphates also doped with acidic ions. Modified clays, such as acid leached bentonites, as such and ion exchanged with acidic ions such as Ce, Zn, Fe, and so on, including pillared synthetic and natural clays.

ACPs also include doped alumina with acidic promoters such as, for example, boehmite doped with phosphate ions, sulphate ions, Rare Earth and transition metal ions, and so on.

The pentasil-type zeolite of the catalyst composition as claimed above may be prepared in any manner as described above.

Use of the catalyst of the invention

The refinery process in which use of the catalyst of the invention in contemplated may be any fluid catalytic cracking process designed to produce light olefins, having up to about 6 carbon atoms per molecule, such as FCC or DCC. The process involves contacting a petroleum feedstock with the catalyst composition of the invention at fluid catalytic cracking conditions, typically comprising a temperature from about 450-780° C., residence time from about 0.01 to 20 seconds, with and without added steam, and a catalyst-to-oil ratio from 1 to 100. The catalyst composition may comprise about 5.0 to about 80 wt % of a mixture of the catalyst composition of the invention and a second fluidized catalytic cracking catalyst composition.

The pentasil zeolites used in the following examples were synthesized and modified with various metals and phosphorous as described above.

### EXAMPLES

### Comparative Example 1

Commercially available ZSM-5 additive (65 wt. % pseudo boehmite alumina and 35 wt. % ZSM-5 zeolite) was calcined and blended with a base catalyst of a formulation 34 wt-% Y zeolite (Re/Y)=2, 13 wt-% (pseudoboehmite) alumina, 12 wt-% binder, and clay to balance. The amount of additive in the blend was 10 wt-%. Absent from the blend was a solid acidic cracking promoter.

### Comparative Example 2

ZSM-5 was mixed with H₃PO₄ solution at pH<3, dried, and calcined at 600° C. for 1 hr. The resulting zeolite (15 wt-% P205) was milled and embedded into a slurry of a peptized (pseudo boehmite) alumina and clay. The slurry was mixed with high shear, dried, and calcined. The final composition was 15 wt-% ZSM-5, 65 wt-% All2O3, and 10 wt-% clay. Also absent from this blend was a solid acidic cracking promoter.

### Example 3

Example 1 was repeated, but instead of 65 wt-% of (pseudo boehmite), alumina in the additive, an acidic cracking promoter of 15 wt-% deeply stabilized, low sodium USY and 15 wt-% modified (pseudo boehmite) alumina was employed. The modified (pseudo boehmite) alumina was prepared by adding 975 g phosphoric acid and 5823 g ReCl₃ (Rare Earth) solution to a heel of H-water. Under stirring, 13700 g Natal (25 wt-% Al2O3) and 10172 g sulphuric acid was added at a fixed pH of 9.5 into the mixture. The slurry was aged at 100° C. for 24 h, filtrated, washed, dried, and calcined.

A summary of catalyst properties and performance for the above Examples is given in the following Table:

Table of catalyst properties and performance

| | E1 | E2 | |
|---|---|---|---|
| | Comparative example | Comparative example | E3 |
| ABD | 0.82 | Na | 0.72 |
| SA BET | 257 | Na | 231 |
| Al2O3 | 73.1 | Na | 36.16 |
| Re2O3 | <0.1 | Na | 6.79 |
| P2O5 | 1.89 | Na | 4.67 |
| Conversion | 63.4 | 76.0 | 78.3 |
| Propylene | 10.2 | 11.1 | 13.3 |
| Butylenes | 8.9 | 9.4 | 10.8 |
| Gasoline | 26.3 | 36.5 | 34.5 |
| Bottoms | 18.4 | 9.1 | 7.9 |

¹Small scale fluidized bed reactor at 540° C. Feed was a long residue with a CCR of 3.2

As is clear from the Table, use of the composition of the invention results in a marked increase in the yield of olefins as compared to use of a conventional composition.

1. A catalyst composition comprising a pentasil-type of zeolite, one or more solid acidic cracking promoters.

2. The catalyst composition of claim 1 comprising a filler and/or binder.

3. The catalyst composition of claim 1 wherein said pentasil zeolite is selected from the group consisting of ITQ-type zeolite, beta zeolite and silicalite.

4. The catalyst composition of claim 1 wherein said pentasil zeolite comprises ZSM-type zeolite.

5. The catalyst composition of claim 1 wherein said pentasil zeolite is doped with a compound comprising a metal ion selected from the group consisting of ions of alkaline earth metals, transition metals, rare earth metals, phosphorous, boron, aluminum, noble metals and combinations thereof.

6. The catalyst composition of claim 1 where in said pentasil zeolite comprises crystals having metals in tetrahedral coordination in said crystals selected from the group consisting of Al, As, B, Be, Co, Cr, Fe, Ga, Hf, In, Mg, Mn, Ni, P, Si, Ti, V, Zn, Zr and mixtures thereof.

7. The catalyst composition of claim 1 wherein said solid acidic cracking promoter is selected from the group consisting of alumina modified by incorporation of acid centers thereon or therein, acidic silica-alumina co-gels, acidic natural or synthetic clays, acidic titania, acidic zirconia, acidic titania-alumina, acidic zeolite materials and co-gels of titania, alumina, zirconia, phosphates, borates, aluminophosphates, tungstates, molybdates and mixtures thereof.

8. The catalyst composition of claim 7 wherein said acid centers are selected from the group consisting of halides, sulfates, nitrates, titanates, zirconates, phosphates, borates, silicates and mixtures thereof.

9. The catalyst composition of claim 7 wherein said solid acidic cracking promoter comprises acidic silica-alumina, titania-alumina, titania/zirconia, alumina/zirconia or aluminum phosphate co-gels modified by the incorporation therein of metal ions or compounds selected from the group consisting of alkaline earth metals, transition metals, rare earth metals and mixtures thereof.

10. The catalyst composition of claim 7 wherein said acidic silica-alumina co-gels have been subjected to hydrothermal treatment.

11. The catalyst composition of claim 7 wherein said acidic natural or synthetic clays have been modified by calcining, steaming, dealumination, desilification, ion exchange, pillaring exfoliation or combinations thereof.

12. The catalyst composition of claim 7 wherein said acid titania, acidic zirconia, or both are doped with sulfates, vanadates, phosphates, tungstates, borates, iron, rare earth metals or mixtures thereof.

13. The catalyst composition of claim 7 wherein said acidic zeolite materials are selected from the group consisting of mordenite, NaY zeolite and USY zeolite that is dealuminated or ion exchanged with transition metals or both.

14. The catalyst composition of claim 13 wherein said transition metal is vanadium.

15. The catalyst composition of claim 1 wherein said solid acidic cracking promoter comprises a co-gel of alumina-aluminum-phosphate or aluminum phosphate that has been doped with an acidic compound.

16. The catalyst composition of claim 1 comprising one or more additional materials selected from the group consisting of particle binders, diluents, fillers and extenders.

17. The catalyst composition of claim 1 wherein the weight ratio of said pentasil-type zeolite to said solid acidic cracking promoter is from about 0.03 to 9.0.

18. The catalyst composition of claim 15 wherein said pentasil-type zeolite is a pentasil type of zeolite that comprises from about 5.0 wt % to about 80 wt % of said composition.

19. The catalyst composition of claim 1 wherein said solid acidic cracking promoter comprises from about 5.0 wt % to about 80 wt % of said composition.

20. The catalyst composition of claim 1 wherein said composition comprises particles having average lengths along their major axis of from about 20 microns to about 200 microns.

21. The catalyst composition of claim 16 wherein said composition comprises particles having average lengths along their major axis of from about 30 microns to about 150 microns.

22. A method of making the catalyst composition of claim 1 wherein an aqueous slurry comprising said pentasil-type zeolite and said solid acidic cracking promoter is prepared and dried.

23. The method of claim 22 wherein separate aqueous slurries of said pentasil-type zeolite and said solid acidic cracking promoter are prepared, mixed together and dried.

24. A method of making the catalyst composition of claim 5 wherein said pentasil-type zeolite is doped by ion exchange with said ions.

25. A method of making the catalyst composition of claim 5 wherein said pentasil-type zeolite is doped by using doped seeds.

26. A method of making the catalyst composition of claim 5 wherein said pentasil-type zeolite is doped by using doped reactants

27. A method of making the catalyst composition of claim 5 wherein said pentasil-type zeolite is doped by using seeds comprising X- or Y-type zeolites that have been ion exchanged with said ions.

28. A method of making the catalyst composition of claim 5 wherein said pentasil-type zeolite is doped by incorporating salts comprising said ions in a reaction mixture comprising the precursor of said pentasil-type zeolite.

29. The method of claim 21 wherein said aqueous slurry is spray dried to obtain catalyst particles having average lengths along their major axis of from about 40 microns to about 100 microns.

30. A method of making the catalyst composition of claim 16 wherein said pentasil-type zeolite has been modified by being ion exchanged with ions selected from the group consisting of ions of alkaline earth metals, transition metals, rare earth metals, phosphorous, boron, aluminum, noble metals and combinations thereof, preparing an aqueous slurry of said acidic cracking promoter and other catalyst ingredients other than said modified pentasil-type zeolite, adding said modified pentasil-type zeolite to said slurry and shaping said slurry, said addition of said modified pentasil-type zeolite being carried out as a final step immediately prior to said shaping.

31. The method of claim 30 wherein said addition of said modified pentasil-type zeolite comprises mixing with said aqueous slurry until said slurry is substantially homogeneous.

32. The method of claim 30 wherein said shaping comprises spray drying.

33. The method of claim 30 wherein NH₄OH is added to said slurry prior to the addition of said modified pentasil-type zeolite to raise the pH of said slurry.

34. The method of claim 30 wherein a pH buffer is added to said slurry prior to the addition of said modified pentasil-type zeolite.

35. The method of claim 34 wherein said pH buffer is selected from the group consisting of aluminum chlorohydrol, phosphate sol or gel, anionic clay, smectite and thermally or chemically modified clay.

36. The method of claim 35 wherein said thermally or chemically modified clay is kaolin clay.

37. A method for preparing the catalyst of claim 1 wherein an aqueous slurry is prepared comprising said solid acidic cracking promoter and precursors of said pentasil-type zeolite comprising silica, alumina and seeds containing one or more metals from the group consisting of rare earth metals, alkaline earth metals and transition group metals, forming said aqueous slurry into shaped bodies and crystallizing said pentasil-type zeolite in situ in said shaped body.

38. A process for producing olefins having up to about 12 carbon atoms per molecule comprising contacting a petroleum feedstock at fluid catalytic cracking conditions with the catalyst composition of claim 1.

39. A process for producing olefins having up to about 6 carbon atoms per molecule comprising contacting a petroleum feedstock at fluid catalytic cracking conditions with the catalyst composition of claim 1.

40. The process of claim 38 wherein said catalyst composition comprises about 5.0 to about 80 wt % of a mixture of said catalyst composition and a second fluidized catalytic cracking catalyst composition.
